(19) United States
(12) Patent Application Publication
(10) Pub. No.: US 2025/0164389 A1
(43) Pub. Date: May 22, 2025

(54) Calibration Method and System

(71) Applicant: Precision Planting LLC, Tremont, IL (US)

(72) Inventor: Michael Molter, Tremont, IL (US)

(21) Appl. No.: 18/840,043

(22) PCT Filed: Mar. 8, 2023
(86) PCT No.: PCT/IB2023/052204

§ 371 Date: Aug. 20, 2024

Related U.S. Application Data
(63) Continuation of application No. PCT/IB2023/052205, filed on Mar. 8, 2023.
(60) Provisional application No. 63/366,673, filed on Jun. 20, 2022.
(60) Provisional application No. 63/366,674, filed on Jun. 20, 2022.
(60) Provisional application No. 63/366,673, filed on Jun. 20, 2022.
(60) Provisional application No. 63/366,674, filed on Jun. 20, 2022.

(51) Int. Cl. G01N 21/27; G01N 21/3577

(52) U.S. Cl. CPC G01N 21/278; G01N 21/3577; G01N 2201/127

(57) ABSTRACT

Aspects generally relate to systems, apparatuses, and methods for calibrating an infrared light reflectance system. Additional aspects relate to liquid compositions adapted for the calibration of an infrared light reflectance sensing system. In accordance with one aspect, a method is provided for calibrating an infrared reflectance device using a liquid composition. The method typically includes providing a composition comprising a plurality of particles and a liquid carrier; emitting an infrared light at the composition using a light source to produce a reflected light; and sensing the reflected light from the composition using a sensor.

### CROSS REFERENCE TO RELATED APPLICATIONS

This application claims priority to U.S. Provisional Application Nos. 63/366,673, filed 20 Jun. 2022, and 63/366,674. Filed 20 Jun. 2022, all of which are incorporated herein by reference in their entireties.

### FIELD OF THE DISCLOSURE

Aspects of the present invention generally relate systems, apparatuses, and methods for calibrating an infrared light reflectance system. Additional aspects of the present invention relate to liquid compositions adapted for the calibration of an infrared light reflectance sensing system.

### BACKGROUND

Periodic soil testing is an important aspect of the agricultural arts. Test results provide valuable information on the chemical makeup of the soil, such as plant-available nutrients and other important properties (e.g., levels of nitrogen, magnesium, phosphorous, potassium, pH, etc.) so that various amendments may be added to the soil to maximize the quality and quantity of crop production.

Recent improvements in soil testing relate to systems and methods for more dynamically assessing various soil properties. One such recent development utilizes unique combinations of sensors, including infrared sensors, to determine the characteristics and properties of the soil. Examples of such recent improvements and developments in soil assessment can be commercially obtained from Precision Planting, LLC.

Infrared light sensors, and particularly sensors configured to sense reflected infrared light, should be periodically calibrated to ensure accuracy and for maintenance purposes. Calibration of such reflected infrared light sensors traditionally includes emitting an infrared light towards a reference puck and sensing the light reflected off the reference puck. A reference puck is typically a solid item having a cylindrical or rectangular shape that has a predetermined reflectivity, which allows for the calibration of an infrared light reflectance sensor.

There is an ongoing need for improvements in agricultural testing as well as for systems, methods, apparatuses, and compositions for improving the accuracy of such testing.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description and brief description of the drawings provided below.

Aspects of the invention are generally directed to systems, apparatuses, and methods for calibrating an infrared light reflectance system and/or device. Additional aspects of the present invention relate to liquid compositions adapted for the calibration of an infrared light reflectance sensing system/device.

In accordance with a further aspect, provided is a calibration composition for calibrating an infrared reflectance device and/or sensor. The calibration composition typically comprises a liquid carrier; a plurality of metal oxide particles; and an acid, wherein the calibration composition has a pH of about 1 to 7.

According to another aspect, provided is a system comprising a light source configured to emit an infrared light; a composition comprising a plurality of particles and a liquid carrier, wherein at least a portion of the plurality of particles is adapted to reflect infrared light; a sensor configured to sense the reflected infrared light; and an apparatus defining an inlet port, an exit port, and a channel extending therebetween. The inlet port is configured to receive the composition and the outlet port is in fluid communication with the inlet port via the channel.

In accordance with one aspect, a method is provided for calibrating an infrared reflectance device using a liquid composition. The method typically includes providing a composition comprising a plurality of particles and a liquid carrier; emitting an infrared light at the composition using a light source to produce a reflected light; and sensing the reflected light from the composition using a sensor.

### BRIEF DESCRIPTION OF THE DRAWINGS

The present invention will become more fully understood from the detailed description and the accompanying drawings, wherein like elements are labeled similarly and in which:

FIG. 1 is a perspective view of an exemplary system according to an aspect of the invention;

FIG. 2 is a perspective view of the apparatus of the system shown in FIG. 1;

FIG. 3 is a cross-sectional schematic of the system shown in FIG. 1;

FIG. 4 is a perspective view of another exemplary system according to an aspect of the invention;

FIG. 5 is a cross-sectional view of the system shown in FIG. 4; and

FIG. 6 is flow chart of a method according to another aspect of the invention.

All drawings are not necessarily to scale. Components numbered and appearing in one figure but appearing un-numbered in other figures are the same unless expressly noted otherwise. A reference herein to a whole figure number which appears in multiple figures bearing the same whole number but with different alphabetical suffixes shall be constructed as a general refer to all of those figures unless expressly noted otherwise.

### DETAILED DESCRIPTION

The features and benefits of the invention are illustrated and described herein by reference to exemplary (“example”) embodiments. This description of exemplary embodiments is intended to be read in connection with the accompanying drawings, which are to be considered part of the entire written description. Accordingly, the disclosure expressly should not be limited to such exemplary embodiments illustrating some possible non-limiting combination of features that may exist alone or in other combinations of features.

In the description of embodiments disclosed herein, any reference to direction or orientation is merely intended for convenience of description and is not intended in any way to limit the scope of the present invention. Relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise.

As used throughout, any ranges disclosed herein are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Aspects of the invention are directed to systems, apparatuses, compositions, and methods for calibrating an infrared light reflectance system. The inventors discovered that significant improvements in the calibration of reflected infrared light sensors can be achieved by replacing a conventional reference puck with certain compositions according the aspects of the invention. Moreover, by utilizing various methods, systems, and apparatuses disclosed herein, improved calibration of reflected infrared light sensors can be achieved while simultaneously increasing the reliability and repeatability of such calibrations. For instance, embodiments of the invention advantageously achieve improved accuracy of calibration, enhanced ease of use, and can be utilized for calibrating broader light spectrums than traditional reference pucks.

In accordance with a first aspect, provided is a calibration composition adapted for use as a reference material for calibrating a sensor configured to sense a reflected infrared light. Preferably, the calibration composition is a liquid composition, which may be aqueous or non-aqueous. The calibration composition may be in the form of a solution, a mixture, or a suspension. In at least one preferred embodiment, the calibration composition is a solution or suspension. For instance, the calibration composition may be in the form of an aqueous solution or aqueous suspension. The calibration composition typically comprises a plurality of particles, an acid, and a liquid carrier. The calibration composition generally has a pH of about 1 to 7.

The plurality of particles is typically adapted to reflect light having wavelengths of about 780 nm to about 1 mm. For example, the plurality of particles may be adapted to reflect light with a wavelength from about 780 nm to about 1 mm, about 780 nm to about 800 μm, about 780 nm to about 600 μm, about 780 nm to about 400 μm, about 780 nm to about 200 μm, about 780 nm to about 100 μm, about 780 nm to about 50 μm, about 780 nm to about 10 μm, about 780 nm to about 5 μm, about 780 nm to about 2 μm; from about 870 nm to about 1 mm, about 870 nm to about 800 μm, about 870 nm to about 600 μm, about 870 nm to about 400 μm, about 870 nm to about 200 μm, about 870 nm to about 100 μm, about 870 nm to about 50 μm, about 870 nm to about 10 μm, about 870 nm to about 5 μm, about 870 nm to about 2 μm; from about 960 nm to about 1 mm, about 960 nm to about 800 μm, about 960 nm to about 600 μm, about 960 nm to about 400 μm, about 960 nm to about 200 μm, about 960 nm to about 100 μm, about 960 nm to about 50 μm, about 960 nm to about 10 μm, about 960 nm to about 5 μm, about 960 nm to about 2 μm; from about 1050 nm to about 1 mm, about 1050 nm to about 800 μm, about 1050 nm to about 600 μm, about 1050 nm to about 400 μm, about 1050 nm to about 200 μm, about 1050 nm to about 100 μm, about 1050 nm to about 50 μm, about 1050 nm to about 10 μm, about 1050 nm to about 5 μm, about 1050 nm to about 2 μm; from about 1.1 μm to about 1 mm, about 1.1 μm to about 800 μm, about 1.1 μm to about 600 μm, about 1.1 μm to about 400 μm, about 1.1 μm to about 200 μm, about 1.1 μm to about 100 μm, about 1.1 μm to about 50 μm, about 1.1 μm to about 10 μm, about 1.1 μm to about 5 μm, about 1.1 μm to about 2 μm, about 1.1 μm to about 1.8 μm, about 1.1 μm to about 1.4 μm, including any range or subrange thereof. Although the plurality of particles are typically adapted to reflect light wavelengths of about 780 nm to about 1 mm, the plurality of particles may also reflect light having wavelengths of below about 780 nm and/or above about 1 mm.

The plurality of particles may have a light reflectivity of about 60% or greater within one or more of the above disclosed wavelength ranges. In some cases, the plurality of particles may be adapted to have about 60% or greater, about 65% or greater, about 70% or greater, about 75% or greater, about 80% or greater, about 90% or greater, about 95% or greater, or about 98% or greater reflectivity within at least one of the above disclosed wavelength ranges, based on U.S. Department of Commerce standards for infrared transmittance SRMs 2053, 2054, 5055, and 5056, which can be found in Standard Reference Materials, NIST Special Publication 260-123, U.S. Department of Commerce/Technology Administration National Institute of Standards and Technology (May 2001). Additional methods for measuring the light reflectivity of a material can be found in W. Budde, Calibration of Reflectance Standards, J. RES. NATL. BUR. STAND. A. PHYS. CHEM., Vol. 80A(4), pgs. 585-595 (July-August 1976). Another method for measuring reflectivity includes using a SmartFirmer™ sensor available from Precision Planting LLC of Tremont, Illinois. For example, the reflectivity of the plurality of particles and/or the calibration composition may be determined using a factory calibrated SmartFirmer™ sensor. Alternatively, the SmartFirmer™ sensor may be calibrated using a reference puck before being used to determine the reflectivity of the plurality of particles and/or the calibration composition.

The plurality of particles of the calibration composition may be selected from microparticles, nanoparticles, or a mixture thereof. For example, the plurality of particles may have an average diameter from about 1 nm to about 25 μm, as determined from the maximum diameter of the particle. As used herein, “average particle diameter,” or “D₅₀ particle size” refers to a particle diameter corresponding to 50% of the particles in a distribution curve in which particles are accumulated in the order of particle diameter from the smallest particle to the largest particle and a total number of accumulated particles is 100%. Similarly, “D₂₅ particle size” refers to a particle diameter corresponding to 25% of the particles in a distribution curve in which particles are accumulated in the order of particle diameter from the smallest particle to the largest particle and a total number of accumulated particles is 100%. Likewise, “D₇₅ particle size” refers to a particle diameter corresponding to 75% of the particles in a distribution curve in which particles are accumulated in the order of particle diameter from the smallest particle to the largest particle and a total number of accumulated particles is 100%. The mean particle diameter may be measured by methods known to those of skill in the art. For example, the mean particle diameter may be as determined with a commercially available particle size analyzer by, e.g., dynamic light scattering, or may be measured using a transmission electron microscope (TEM) or a scanning electron microscope (SEM). When determined by TEM or SEM, an average longest dimension of a particle may be used.

In some instances, the plurality of particles may be microparticles and, e.g., have an average particle diameter of about 1 to about 25 μm, about 1 to about 20 μm, about 1 to about 15 μm, about 1 to about 10 μm, about 1 to about 5 μm; from about 5 to about 25 μm, about 5 to about 20 μm, about 5 to about 15 μm, about 5 to about 10 μm; from about 10 to about 25 μm, about 10 to about 20 μm, about 10 to about 15 μm, including any range or subrange thereof, as determined from the maximum diameter of the particle. In some embodiments, the plurality of particles may be nanoparticles and have an average particle size, e.g., from about 1 nm to about 1 μm, as determined from the maximum diameter of the particle. For instance, the plurality of particles may have an average particle size from about 10 nm to about 1 μm, about 1 to about 900 nm, about 10 to about 800 nm, about 10 to about 700 nm, about 10 to about 600 nm, about 10 to about 550 nm, about 10 to about 500 nm, about 10 to about 450 nm; from about 50 nm to about 1 μm, about 50 to about 900 nm, about 50 to about 800 nm, about 50 to about 700 nm, about 50 to about 600 nm, about 50 to about 550 nm, about 50 to about 500 nm, about 50 to about 450 nm; from about 100 nm to about 1 μm, about 100 to about 900 nm, about 100 to about 800 nm, about 100 to about 700 nm, about 100 to about 600 nm, about 100 to about 550 nm, about 100 to about 500 nm, about 100 to about 450 nm; from about 200 nm to about 1 μm, about 200 to about 900 nm, about 200 to about 800 nm, about 200 to about 700 nm, about 200 to about 600 nm, about 200 to about 550 nm, about 200 to about 500 nm, about 200 to about 450 nm; from about 300 nm to about 1 μm, about 300 to about 900 nm, about 300 to about 800 nm, about 300 to about 700 nm, about 300 to about 600 nm, about 300 to about 550 nm, about 300 to about 500 nm, about 300 to about 450 nm, or any range or subrange thereof, as determined from the maximum diameter of the particle.

Additionally or alternatively, the plurality of the particles may have a D₂₅ particle size that is smaller than the average particle diameter (D₅₀ particle size) by about 40% or less. For example, the D₂₅ particle size may be smaller than the average particle diameter by about 30% or less, about 25% or less, about 20% or less, about 15% or less, about 10% or less, or about 5% or less. The D₇₅ particle size of the plurality of particles may be larger than the average particle diameter (D₅₀ particle size) by about 40% or less. In some instances, D₇₅ particle size of the plurality of particles may be larger than the average particle diameter by about 30% or less, about 25% or less, about 20% or less, about 15% or less, about 10% or less, or about 5% or less.

The plurality of particles may comprise one or more metal oxide(s), such as those selected from aluminum oxides, magnesium oxides, titanium oxides, zinc oxides, cerium oxides, and a combination of two or more thereof. In some embodiment, at least one of the plurality of metal oxides is selected from aluminum oxides, titanium oxides, and a combination of two or more thereof. For example, the plurality of metal oxide particles may comprise titanium dioxide, alumina, or a combination thereof. The plurality of metal oxides may be selected from CeO₂, TiO₂, MgO, Al₂O₃, ZnO, and a combination of two or more thereof. In some embodiments, the plurality of particles may comprise two or more, three or more, four or more, or five or more metal oxides. For instance, the calibration compositions may comprise two or more metal oxides selected from aluminum oxides, magnesium oxides, titanium oxides, zinc oxides, cerium oxides, and a combination of two or more thereof.

The plurality of particles may comprise at least two metal oxides selected from aluminum oxides, magnesium oxides, titanium oxides, zinc oxides, cerium oxides, and a combination of two or more thereof, wherein the two metal oxides are in a weight ratio of about 1:10 to about 10:1. For example, the calibration composition may comprise at least two metal oxides, with two metal oxides in a weight ratio of from about 1:10 to about 10:1, about 1:7 to about 10:1, about 1:5 to about 10:1, about 1:3 to about 10:1, about 1:1 to about 10:1; from about 1:10 to about 7:1, about 1:7 to about 7:1, about 1:5 to about 7:1, about 1:3 to about 7:1, about 1:1 to about 7:1; from about 1:10 to about 5:1, about 1:7 to about 5:1, about 1:5 to about 5:1, about 1:3 to about 5:1, about 1:1 to about 5:1; from about 1:10 to about 3:1, about 1:7 to about 3:1, about 1:5 to about 3:1, about 1:3 to about 3:1, about 1:1 to about 3:1; from about 1:10 to about 1:1, about 1:7 to about 1:1, about 1:5 to about 1:1, about 1:3 to about 1:1, or about 1:1, including any range or subrange thereof.

The plurality of particles may be present in the calibration composition in an amount from about 0.05 to about 24 wt. %, based on the total weight of the calibration composition. For example, the calibration composition may include a plurality of particles in an amount from about 0.05 to about 24 wt. %, about 0.05 to about 20 wt. %, about 0.05 to about 16 wt. %, about 0.05 to about 12 wt. %, about 0.05 to about 10 wt. %, about 0.05 to about 8 wt. %, about 0.05 to about 6 wt. %, about 0.05 to about 5 wt. %, about 0.05 to about 4 wt. %, about 0.05 to about 3 wt. %, about 0.05 to about 2 wt. %, about 0.05 to about 1 wt. %; from about 0.1 to about 24 wt. %, about 0.1 to about 20 wt. %, about 0.1 to about 16 wt. %, about 0.1 to about 12 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 24 wt. %, about 0.5 to about 20 wt. %, about 0.5 to about 16 wt. %, about 0.5 to about 12 wt. %, about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 24 wt. %, about 1 to about 20 wt. %, about 1 to about 16 wt. %, about 1 to about 12 wt. %, about 1 to about 10 wt. %, about 1 to about 8 wt. %, about 1 to about 6 wt. %, about 1 to about 5 wt. %, about 1 to about 4 wt. %, about 1 to about 3 wt. %; from about 3 to about 24 wt. %, about 3 to about 20 wt. %, about 3 to about 16 wt. %, about 3 to about 12 wt. %, about 3 to about 10 wt. %, about 3 to about 8 wt. %, about 3 to about 6 wt. %; from about 6 to about 24 wt. %, about 6 to about 20 wt. %, about 6 to about 16 wt. %, about 6 to about 12 wt. %, about 6 to about 10 wt. %, about 6 to about 8 wt. %; from about 8 to about 24 wt. %, about 8 to about 20 wt. %, about 8 to about 16 wt. %, about 8 to about 12 wt. %, about 8 to about 10 wt. %; from about 10 to about 24 wt. %, about 10 to about 20 wt. %, about 10 to about 16 wt. %, about 10 to about 12 wt. %, including any range or subrange thereof, based on the total weight of the calibration composition.

The calibration composition typically includes one or more acid(s). The one or more acid(s) may be chosen from hydrochloric acid, hydrobromic acid, hydroiodic acid, nitric acid, perchloric acid, sulfuric acid, l-glutamic acid, lactic acid, malic acid, succinic acid, acetic acid, formic acid, hydrogen sulfide, trichloracetic acid, fumaric acid, tartaric acid, citric acid, l-glutamic hydrochloride, maleic acid, and combinations of two or more thereof. In some embodiments, at least one acid is selected from weak acids or acids other than strong acids. For instance, the one or more acid(s) may comprises l-glutamic acid, lactic acid, hydrochloric acid, malic acid, succinic acid, acetic acid, formic, hydrogen sulfide, trichloracetic acid, fumaric acid, tartaric acid, citric acid, l-glutamic hydrochloride, maleic acid, and combinations of two or more thereof.

The amount of acid(s) present in the calibration composition may be based on the desired pH of the calibration composition. In some embodiments, the one or more acid(s) is present in the calibration composition in an amount from about 0.05 to about 15 wt. %, based on the total weight of the calibration composition. For example, the calibration composition may have one or more acid(s) in an amount from about 0.05 to about 15 wt. %, about 0.05 to about 10 wt. %, about 0.05 to about 7 wt. %, about 0.05 to about 5 wt. %, about 0.05 to about 4 wt. %, about 0.05 to about 3 wt. %, about 0.05 to about 2 wt. %, about 0.05 to about 1 wt. %; from about 0.5 to about 15 wt. %, about 0.5 to about 10 wt. %, about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 15 wt. %, about 1 to about 10 wt. %, about 1 to about 7 wt. %, about 1 to about 5 wt. %, about 1 to about 4 wt. %, about 1 to about 3 wt. %, about 1 to about 2 wt. %; from about 3 to about 15 wt. %, about 3 to about 10 wt. %, about 3 to about 7 wt. %, about 3 to about 5 wt. %; from about 5 to about 15 wt. %, about 5 to about 10 wt. %, about 5 to about 7 wt. %; from about 7 to about 15 wt. %, about 7 to about 10 wt. %, or any range and subrange thereof, based on the total weight of the calibration composition.

Preferably, the pH of the calibration composition may be from about 1 to 7. For some embodiments, the calibration composition has a pH of about 1 to about 6, about 1 to about 5, about 1 to about 4, about 1 to about 3, about 1 to about 2; from about 2 to 7, about 2 to about 6, about 2 to about 5, about 2 to about 4, about 2 to about 3; from about 3 to 7, about 3 to about 6, about 3 to about 5, about 3 to about 4, or any range and subrange thereof. The calibration composition may be formulated to have a pH of about 3 to 7, for example, when further comprising a stabilizing agent. In some embodiments, the calibration composition may have a pH from about 3 to 7, about 4 to 7, about 5 to 7; from about 3 to about 6.5, about 4 to about 6.5, about 5 to about 6.5; from about 3 to about 6, about 4 to about 6, about 5 to about 6; from about 3 to about 5, about 4 to about 5; or about 5 to about 6, including ranges and subranges thereof.

The calibration composition may comprise a stabilization agent that is adapted to suspend and/or promote suspension of a plurality of particles. In at least one preferred embodiment, the stabilization agent comprises poly(methacrylic acid). The stabilization agent may, optionally, be chosen from amino acids, thiols, hydrophilic polymers, hydrophobic polymers, amphiphilic polymers, surfactants, target-specific ligands, and combinations of two or more thereof. Non-limiting examples of amino acids that may be suitable as a stabilization agent include aspartic acid, leucine, lysine, or a combination thereof. Examples of thiols that may be suitable as stabilization agents include aminothiol, thioglycerol, thioglycine, thiolactic acid, thiomalic acid, thiooctic acid, thiosilane, or a combination thereof. Examples of hydrophilic polymers include polyvinylpyrollidone, polyethyleneglycol and copolymers and blends comprising at least one of the monomers which form the said polymers. Additionally polymers worth mentioning that may be suitable as stabilization agents include polyurethanes, acrylic polymers, epoxies, silicones and fluorosilicones.

The calibration composition typically includes a liquid carrier. Although the liquid carrier preferably comprises water, the liquid carrier may, additionally or alternatively, comprise a polyol, a monoalcohol, a fatty acid, an ester, an ether, or a combination of two or more thereof. Examples of monoalcohols include short chain monoalcohols having 2 to 8 carbons and fatty alcohols having a carbon chain of 9 to 32 carbons. Non-limiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, and a mixture thereof. Non-limiting examples of fatty acids include diacids, triacids, and other multiple acids as well as salts of these fatty acids, such as those chosen from lauric acid, palmitic acid, stearic acid, behenic acid, arichidonic acid, oleic acid, isostearic acid, sebacic acid, and a mixture thereof.

The calibration composition may contain the one or more liquid carrier(s) in an amount that may vary, but is typically from about 20 to about 99 wt. %, based on the total weight of the calibration composition. For example, the calibration composition may comprise liquid carrier(s) in an amount from about 20 to about 99 wt. %, about 20 to about 95 wt. %, about 20 to about 90 wt. %, about 20 to about 80 wt. %, about 20 to about 70 wt. %, about 20 to about 60 wt. %, about 20 to about 50 wt. %, about 20 to about 40 wt. %; about 30 to about 99 wt. %, about 30 to about 95 wt. %, about 30 to about 90 wt. %, about 30 to about 80 wt. %, about 30 to about 70 wt. %, about 30 to about 60 wt. %, about 30 to about 50 wt. %, about 30 to about 40 wt. %; about 40 to about 99 wt. %, about 40 to about 95 wt. %, about 40 to about 90 wt. %, about 40 to about 80 wt. %, about 40 to about 70 wt. %, about 40 to about 60 wt. %, about 40 to about 50 wt. %; about 50 to about 99 wt. %, about 50 to about 95 wt. %, about 50 to about 90 wt. %, about 50 to about 80 wt. %, about 50 to about 70 wt. %, about 50 to about 60 wt. %; about 60 to about 99 wt. %, about 60 to about 95 wt. %, about 60 to about 90 wt. %, about 60 to about 80 wt. %, about 60 to about 70 wt. %; about 70 to about 99 wt. %, about 70 to about 95 wt. %, about 70 to about 90 wt. %, about 70 to about 80 wt. %; about 80 to about 99 wt. %, about 80 to about 95 wt. %, about 80 to about 90 wt. %; about 90 to about 99 wt. %, about 90 to about 95 wt. %; or about 95 to about 99 wt. %, including ranges and subranges thereof, based on the total weight of the calibration composition.

In accordance with another aspect, a system 100 is provided. System 100 may be configured to calibrate a sensor 120 configured to sense a reflected light. System 100 may include a light source 110, a sensor 120, and an apparatus 130 having a channel 136 adapted to receive a composition (such as, a calibration composition disclosed herein). As a brief overview, system 100 is preferably configured such that a light emitted from light source 110 and reflected off the composition in the channel 136 of apparatus 130 is sensed/detected by sensor 120. For instance, one or more of light source 110, sensor 120, and apparatus 130 may be positioned or positionable so that light reflected off the composition is sensed by sensor 120.

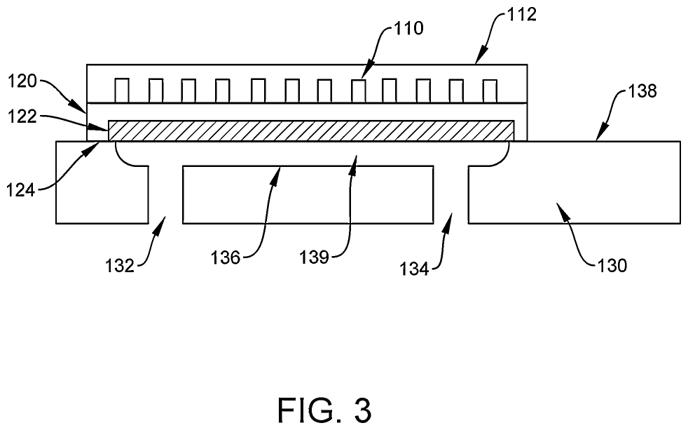

Light source 110 may be any suitable device configured to produce and emit a light having parameters in accordance with aspects of the invention. For example, the light source may be a light emitting diode (“LED”) light source or the like. As seen in FIG. 3A, system 110 may comprise a plurality of light sources 110 that are part of a light source device 112. Preferably, the light source is configured to produce and emit an infrared light. For example, light source 110 may emit light having a wavelength of about 780 nm to about 1 mm. For example, in some instances, the emitted light may have a wavelength of from about 780 nm to about 1 mm, about 780 nm to about 800 μm, about 780 nm to about 600 μm, about 780 nm to about 400 μm, about 780 nm to about 200 μm, about 780 nm to about 100 μm, about 780 nm to about 50 μm, about 780 nm to about 10 μm, about 780 nm to about 5 μm, about 780 nm to about 2 μm; from about 870 nm to about 1 mm, about 870 nm to about 800 μm, about 870 nm to about 600 μm, about 870 nm to about 400 μm, about 870 nm to about 200 μm, about 870 nm to about 100 μm, about 870 nm to about 50 μm, about 870 nm to about 10 μm, about 870 nm to about 5 μm, about 870 nm to about 2 μm; from about 960 nm to about 1 mm, about 960 nm to about 800 μm, about 960 nm to about 600 μm, about 960 nm to about 400 μm, about 960 nm to about 200 μm, about 960 nm to about 100 μm, about 960 nm to about 50 μm, about 960 nm to about 10 μm, about 960 nm to about 5 μm, about 960 nm to about 2 μm; from about 1050 nm to about 1 mm, about 1050 nm to about 800 μm, about 1050 nm to about 600 μm, about 1050 nm to about 400 μm, about 1050 nm to about 200 μm, about 1050 nm to about 100 μm, about 1050 nm to about 50 μm, about 1050 nm to about 10 μm, about 1050 nm to about 5 μm, about 1050 nm to about 2 μm; from about 1.1 μm to about 1 mm, about 1.1 μm to about 800 μm, about 1.1 μm to about 600 μm, about 1.1 μm to about 400 μm, about 1.1 μm to about 200 μm, about 1.1 μm to about 100 μm, about 1.1 μm to about 50 μm, about 1.1 μm to about 10 μm, about 1.1 μm to about 5 μm, about 1.1 μm to about 2 μm, about 1.1 μm to about 1.8 μm, about 1.1 μm to about 1.4 μm, including any range or subrange thereof. In at least one preferred embodiment, light source 110 is configured to emit an infrared light source. Light source 110 may, in some instance, be configured to emit a light having a wavelength below about 780 nm and/or above about 1 mm.

Light source 110 may be configured to emit a light having a radiant power density from about 1 to about 70 mW/cm². For example, the light may have a radiant power density of from about 1 to about 70 mW/cm, about 1 to about 50 mW/cm, about 1 to about 30 mW/cm; from about 10 to about 70 mW/cm; about 10 to about 50 mW/cm, about 10 to about 30 mW/cm; from about 20 to about 70 mW/cm, about 20 to about 50 mW/cm, about 20 to about 30 mW/cm; from about 30 to about 70 mW/cm, about 30 to about 50 mW/cm; from about 40 to about 70 mW/cm, about 40 to about 50 mW/cm; from about 50 to about 70 mW/cm, or about 60 to about 70 mW/cm.

In some embodiments, light source 110 has power from about 5 to about 200 mW. For example, light source 110 may be from about 5 to about 180 mW, about 5 to about 160 mW, about 5 to about 140 mW, about 5 to about 120 mW, about 5 to about 100 mW, about 5 to about 80 mW, about 5 to about 60 mW; about 5 to about 50 mW, about 5 to about 40 mW, 10 to about 180 mW, about 10 to about 160 mW, about 10 to about 140 mW, about 10 to about 120 mW, about 10 to about 100 mW, about 10 to about 80 mW, about 10 to about 60 mW; about 10 to about 50 mW, about 10 to about 40 mW, 20 to about 180 mW, about 20 to about 160 mW, about 20 to about 140 mW, about 20 to about 120 mW, about 20 to about 100 mW, about 20 to about 80 mW, about 20 to about 60 mW; about 20 to about 50 mW, about 20 to about 40 mW, about 30 mW or any range or subrange thereof.

In some embodiments, light source 110 has power from about 5 to about 200 W. For example, light source 110 may be from about 5 to about 180 W, about 5 to about 160 W, about 5 to about 140 W, about 5 to about 120 W, about 5 to about 100 W, about 5 to about 80 W, about 5 to about 60 W; from about 20 to about 180 W, about 20 to about 160 W, about 20 to about 140 W, about 20 to about 120 W, about 20 to about 100 W, about 20 to about 80 W, about 20 to about 60 W; from about 50 to about 180 W, about 50 to about 160 W, about 50 to about 140 W, about 50 to about 120 W, about 50 to about 100 W, about 50 to about 80 W; from about 80 to about 180 W, about 80 to about 160 W, about 80 to about 140 W, about 80 to about 120 W, about 80 to about 100 W; from about 110 to about 180 W, about 110 to about 160 W, about 110 to about 140 W; from about 140 to about 180 W, or any range or subrange thereof.

System 100 may include at least one sensor 120 configured to sense/detect a reflected light and, preferably, a reflected infrared light. Sensor 120 may be selected based on light source 110 and/or the expected reflected light. In one embodiment, light source 110 and sensor 120 may be features and/or parts of a single device.

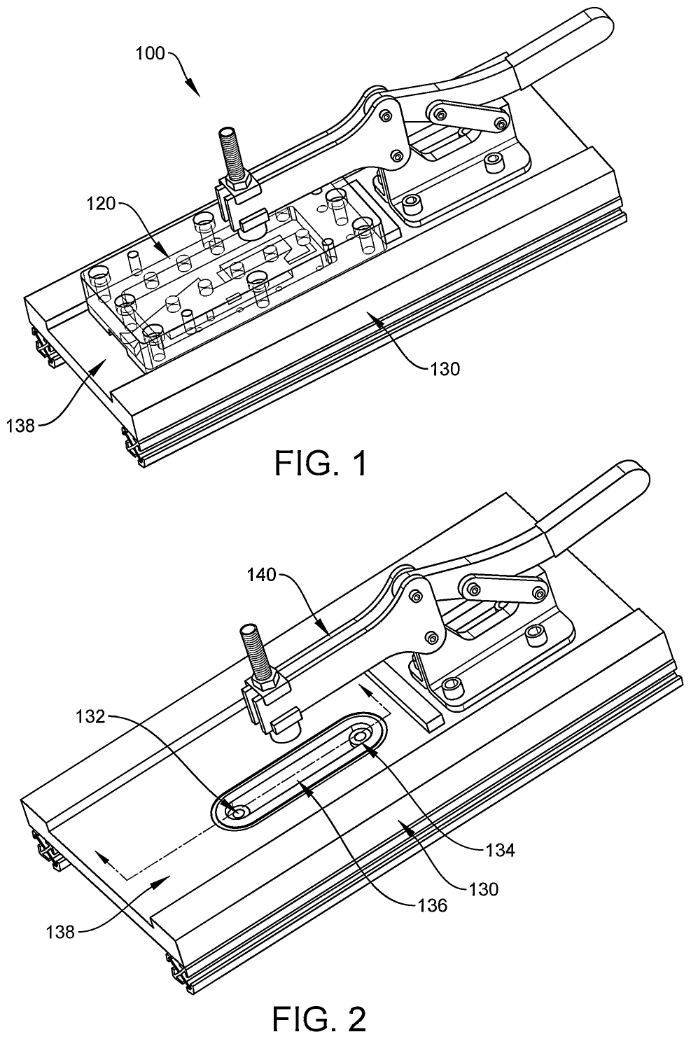

Referring to FIGS. 1-3, apparatus 130 of system 100 may have a surface 138 that defines an inlet port 132, an exit port 134, and a channel 136 extending therebetween. Channel 136 typically extends from inlet port 132 to exit port 134, thereby enabling a fluid (e.g., a calibration composition) to flow from inlet port 132 to export 134. Although channel 136 is depicted in FIG. 2 as being delineated by a concave curvature extending from inlet port 132 to exit port 134, in other embodiments channel 136 may be delineated as a rectangular concavity, pentagonal concavity, hexagonal concavity, heptagonal concavity, octagonal concavity, or other geometric concavity extending between inlet port 132 and 136. As seen in FIG. 2, in at least one embodiment, channel 136 extends in a linear direction between inlet port 132 and exit port 134.

Inlet port 132 is typically in fluid communication with a source of a composition (e.g., a calibration composition) disclosed herein. For example, inlet port 132 may be coupled to a conduit, such as a pipe, a hose, a tube, or the like that is coupled to or in fluid communication with the source of the composition, such that inlet port 132 is in fluid communication with the container containing the composition. Additionally or alternatively, exit port 134 may be in fluid communication with a waste container via a conduit, such as a pipe, a hose, a tube, or the like. In some embodiments, however, exit port 134 may be in fluid communication with the source of the composition and/or a recycle stream, such that the composition can be recycled after flowing through channel 136.

Apparatus 130 may be formed from a material that is compatible with the compositions (e.g., the calibration compositions) disclosed herein. In some embodiments, apparatus 130 is formed from metal(s), metal alloy(s), plastic(s) and/or polymer resin(s), ceramic(s), or a combination of two or more thereof. Apparatus 130 may, in at least one embodiment, be in the form of a substrate.

System 100 is preferably configured such that light source 110 is positioned or positionable relative to sensor 120. Generally, light source 110 and/or sensor 120 may be positioned relative to each other based on the angle of incidence law (sometimes referred to the law of reflection), where φᵢₙ=φ_{ref}. In other words, the angle of incidence (φᵢₙ) between the emitted infrared light and a normal line (i.e., a line perpendicular to the composition at the point of incidence) is equal to the angle of reflection (φ_{ref}) between the normal line and the reflect infrared light.

For instance, light source 110 may be positioned so that the angle of the emitted infrared light relative to the composition yields a light reflected off the composition at an angle that enables sensor 120 to sense the reflected light and not sense transmitted light. In some embodiments, light source 110 and/or sensor 120 are positioned such that the angle of incidence and/or angle of reflection is from about 10 to about 80°, about 20 to about 70°, about 30 to about 60°, relative to the normal line. In further embodiments, light source 110 and/or sensor 120 may be positioned so the angle of incidence and/or angle of reflection is from about 20 to about 70°, about 30 to about 70°, about 40 to about 70°, about 45 to about 70°, about 50 to about 70°, about 60 to about 70°; from about 20 to about 60°, about 30 to about 60°, about 40 to about 60°, about 45 to about 60°, about 50 to about 60°; from about 20 to about 50°, about 30 to about 50°, about 40 to about 50°, about 45 to about 50°, or any range thereof, relative to the normal line.

System 100 may also be configured such that light source 110 is positioned or positionable relative to surface 138 of apparatus 130, such that the light is emitted at an angle of about 90° relative to the composition when flowing through channel 136. In some embodiments, however, light source 110 is positioned relative to apparatus 130, such that the light is emitted at an angle of from about 20 to about 160°, about 20 to about 140°, about 20 to about 120°, about 20 to about 110°, about 20 to about 100°; from about 40 to about 160°, about 40 to about 140°, about 40 to about 120°, about 40 to about 110°, about 40 to about 100°; from about 60 to about 160°, about 60 to about 140°, about 60 to about 120°, about 60 to about 110°, about 60 to about 100°; from about 70 to about 160°, about 70 to about 140°, about 70 to about 120°, about 70 to about 110°, about 70 to about 100°; from about 80 to about 160°, about 80 to about 140°, about 80 to about 120°, about 80 to about 110°, about 80 to about 100°, about 85 to about 95°, or any range or subrange thereof, relative to the composition when flowing through channel 136.

Additionally or alternatively, sensor 120 may be positioned or positionable relative to surface 138 of apparatus 130, such that the reflected light is received at an angle at about 90° relative to the composition when flowing through channel 136. In some embodiments, however, sensor 120 is positioned relative to apparatus 130, such that the reflected light is received by sensor 120 at an angle of from about 20 to about 160°, about 20 to about 140°, about 20 to about 120°, about 20 to about 110°, about 20 to about 100°; from about 40 to about 160°, about 40 to about 140°, about 40 to about 120°, about 40 to about 110°, about 40 to about 100°; from about 60 to about 160°, about 60 to about 140°, about 60 to about 120°, about 60 to about 110°, about 60 to about 100°; from about 70 to about 160°, about 70 to about 140°, about 70 to about 120°, about 70 to about 110°, about 70 to about 100°; from about 80 to about 160°, about 80 to about 140°, about 80 to about 120°, about 80 to about 110°, about 80 to about 100°, about 85 to about 95°, or any range or subrange thereof, relative to the composition when flowing through channel 136.

With reference to FIG. 1, a surface 124 of sensor 120 may be positioned to contact a surface 138 of apparatus 130. In some embodiments, when sensor 120 is positioned in contact with surface 138, a passageway 139 is formed between channel 136 and a surface of sensor 120. Passageway 139 may be delineated by a conduit that is collectively formed by surface 124 and/or lens 122 of sensor 120 and the surface defining channel 136. In embodiments where channel 136 is formed by surface 138 of apparatus 130 and one or more walls, the conduit delineating passageway 139 may collectively be formed from the surface 138 of apparatus 130, sensor 120, and one or more walls coupled to apparatus 130 and/or sensor 120. Passageway 139 may fluidically couple inlet port 132 to exit port 134, e.g., via extending from inlet port 132 to exit port 134.

The surface 124 of sensor 120 preferably includes the lens 122 of sensor 120. In at least one preferred embodiment, sensor 120 may be positioned to be in intimate contact with a composition (e.g., calibration composition) disclosed herein when the composition flows through channel 136. For instance, sensor 120 may be positioned to form passageway 139 with channel 136, such that when the composition flows through passageway 139 formed between channel 136 and sensor 120 the composition is in inmate contact (e.g., direct contact) with both the channel 136 and the lens 122 of sensor 120.

In at least one embodiment, however, the composition is separated from sensor 120 (e.g., the lens of the sensor) by a gap when flowing through channel 136. The gap between the composition and lens 122 of sensor 120 may be from about 0.5 mm to about 50 mm, when the composition is flowing through channel 136. For example, the gap between the composition, when flowing through channel 136, and lens 122 of sensor 120 may be about 0.5 mm to about 50 mm, about 0.5 mm to about 40 mm, about 0.5 mm to about 30 mm, about 0.5 mm to about 20 mm, about 0.5 mm to about 10 mm, about 0.5 mm to about 5 mm, about 0.5 mm to about 3 mm, about 0.5 mm to about 1 mm; from about 1 mm to about 50 mm, about 1 mm to about 40 mm, about 1 mm to about 30 mm, about 1 mm to about 20 mm, about 1 mm to about 10 mm, about 1 mm to about 5 mm, about 1 mm to about 3 mm; from about 3 mm to about 50 mm, about 3 mm to about 40 mm, about 3 mm to about 30 mm, about 3 mm to about 20 mm, about 3 mm to about 10 mm, about 3 mm to about 5 mm; from about 10 mm to about 50 mm, about 10 mm to about 40 mm, about 10 mm to about 30 mm, about 10 mm to about 20 mm; from about 20 mm to about 50 mm, about 20 mm to about 40 mm, about 20 mm to about 30 mm, or any range or subrange thereof. The gap between the composition and lens 122 when the composition is flow through channel 136 may comprise air, an inert gas (e.g., nitrogen, helium, etc.), glass, or plastic. In some embodiments, the composition is separated from sensor 120 (e.g., the lens 122 of the sensor 120) by on by air when flowing through channel 136.

System 100 may include a clamp 140 coupled to apparatus 130. Clamp 140 is preferably configured to maintain light source 110 and/or sensor 120 in a position relative to apparatus 130. For example, as illustrated in FIG. 1, clamp 140 may be configured to maintain the position of sensor 120 relative to apparatus 130 by biasing sensor 120 toward surface 138 of apparatus 130.

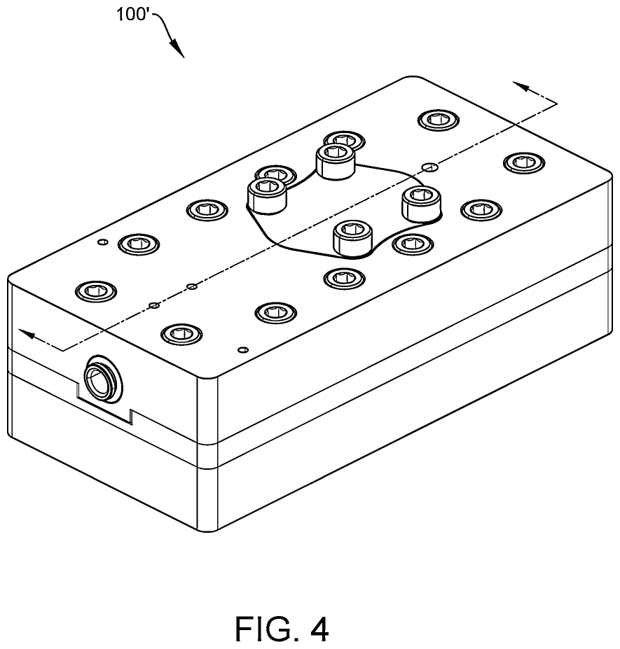

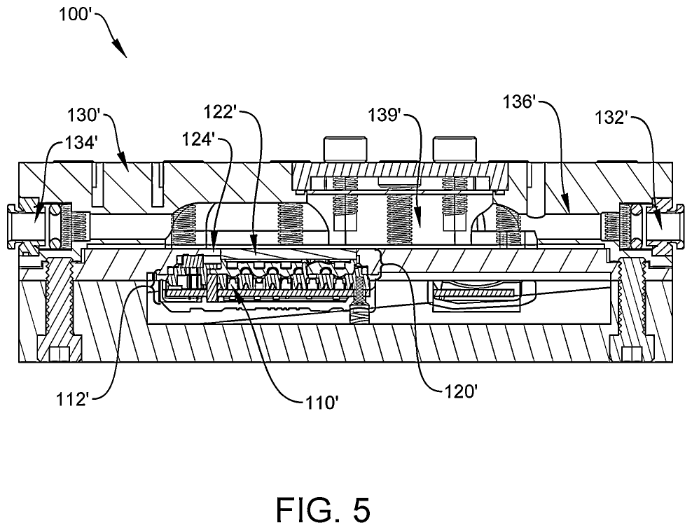

Referring to FIGS. 4 and 5, provided is another exemplary system 100′ according to an aspect of the invention. System 100′ contains similar features as system 100 and, thus, similar reference numbers are utilized. For purposes of brevity, certain features found in both system 100 and system 100′ may be omitted or not discussed again with respect to system 100′.

As a general overview, system 100′ is configured such that a light emitted from light source 110′ of light source device 112′ and reflected off the composition in the channel 136′ of apparatus 130′ is sensed/detected by sensor 120′. Although channel 136′ may be formed by a concavity defined by a surface of apparatus 130′, channel 136′ may be formed by one or more surfaces of apparatus 130′ and one or more walls extending from and/or coupled to apparatus 130′. Channel 136′ preferably extends from inlet port 132′ to exit port 134′, such that inlet port 132′ is in fluidic communication with exit port 134′.

System 100′ is preferably configured such that a passageway 139′ may be formed by channel 136′ and a surface of sensor 120′. For example, channel 136′ and a surface of sensor 120′ may collectively form a conduit that delineates passageway 139′, which may fluidically couple inlet port 132′ and exit port 134′. When channel 136′ is formed by one or more surfaces of apparatus 130′ and one or more walls, the conduit delineating passageway 139′ may be collectively formed from the one or more surfaces of apparatus 130′, sensor 120′, and one or more walls coupled to apparatus 130′ and/or sensor 120′.

Similar to system 100, passageway 139′ may be formed by a surface and/or lens 122′ of sensor 120′ and channel 136′ of apparatus 130′. In at least one preferred embodiment, sensor 120′ may be positioned to be in intimate contact with a composition (e.g., calibration composition) disclosed herein when the composition flows through channel 136′. For instance, sensor 120′ may be positioned to form a passageway 139′ with channel 136′, such that when the composition flows through the passageway 139′ formed between channel 136′ and sensor 120′ the composition is in inmate contact (e.g., direct contact) with both the channel 136′ and the lens 122′ of sensor 120′.

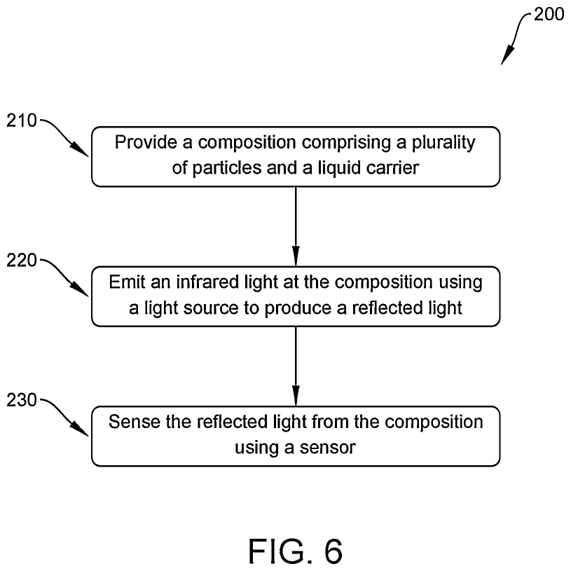

According to a further aspect, a method 200 is provided for calibrating an infrared reflectance device (e.g., sensor/detector) using a liquid composition. Referring to FIG. 6 method 200 typically comprises providing a composition comprising a plurality of particles and a liquid carrier (see step 210); emitting an infrared light at the composition using a light source to produce a reflected light (see step 220); and sensing the reflected light from the composition using a sensor (see step 230). Method 200 may employ any of the systems, apparatuses, and/or compositions disclosed herein and/or one or more components thereof. For example, method 200 may use system 100 and/or apparatus 130, which are described above.

In step 210, a composition is provided comprising a plurality of particles and a liquid carrier. In some instances, the step of providing the composition includes obtaining the components and/or ingredients of the composition and, optionally, mixing the components and/or ingredients together. The compositions employed by method 200 may be any of the calibration compositions discussed herein. Preferably, the compositions are adapted for use as a reference material for calibrating a sensor configured to sense a reflected infrared light. For instance, the composition of method 200 may comprise a plurality of particles, an acid, and a liquid carrier, wherein the calibration composition generally has a pH of about 1 to 7.

In some embodiments, method 200 provides the composition by causing and/or permitting the composition to flow through an inlet port (e.g., inlet port 132) of an apparatus (e.g., apparatus 130). The composition may then flow through a channel (e.g., channel 136) delineated by the apparatus and/or through a passageway formed by the channel and a sensor (e.g., the lens of sensor 120) to an exit port (e.g., exit port 134). In some embodiments, the composition makes intimate contact with the sensor and/or the lens thereof when flowing through the channel. In further embodiments, the composition is separated from the sensor and/or the lens thereof by a gap when flowing through the channel. In at least one embodiment, the composition is separated from the sensor and/or a lens thereof only by air when flowing through the channel.

Additionally or alternatively, method 200 may include diluting the composition to obtain a diluted compositions having a reduced concentration of the plurality of particles. In some embodiments, the composition may be diluted by combining a stream obtained from the composition source with a stream comprising a water concentration greater than the stream from the composition source. In other embodiments, however, a batch of the diluted composition may be obtained and subsequently introduced as the source of the composition. The composition may be diluted to obtain a concentration for the plurality of particles of about 0.1 to about 20 wt. %, preferably about 0.1 to about 15 wt. %, or preferably about 0.1 to about 10 wt. %, based on the total weight of the composition.

In step 220, an infrared light is emitted toward the composition (e.g., the calibration compositions discussed herein) using a light source to produce a reflected infrared light. The reflected infrared light may be produced by at least a portion of the emitted infrared light being reflected off the composition.

In step 230, the reflected infrared light is sensed/detected by the sensor. Method 200 may include positioning the light source and/or the sensor such that infrared light reflecting off the composition is sensed by the sensor. As discussed above, the light source and/or the sensor may be positioned relative to each other based on the angle of incidence law (sometimes referred to the law of reflection), where φᵢₙ=φ_{ref}. For example, the light source and/or the sensor are positioned such that the angle of incidence and/or angle of reflection is from about 10 to about 80°, about 20 to about 70°, about 30 to about 60°, relative to the normal line. Additionally or alternatively, the light source and/or the sensor may be positioned relative to apparatus 130, such that the light is emitted at an angle of from about 20 to about 160°, about 20 to about 140°, about 20 to about 120°, about 20 to about 110°, about 20 to about 100°; from about 40 to about 160°, about 40 to about 140°, about 40 to about 120°, about 40 to about 110°, about 40 to about 100°; from about 60 to about 160°, about 60 to about 140°, about 60 to about 120°, about 60 to about 110°, about 60 to about 100°; from about 70 to about 160°, about 70 to about 140°, about 70 to about 120°, about 70 to about 110°, about 70 to about 100°; from about 80 to about 160°, about 80 to about 140°, about 80 to about 120°, about 80 to about 110°, about 80 to about 100°, about 85 to about 95°, or any range or subrange thereof, relative to the composition when flowing through the channel delineated by the apparatus.

### Examples

The following are nonlimiting examples.
Example 1—a method for calibrating an infrared reflectance device using a liquid composition, the method comprising: providing a composition comprising a plurality of particles and a liquid carrier; emitting an infrared light at the composition using a light source to produce a reflected light; and sensing the reflected light from the composition using a sensor.
Example 2—the method according to Example 1, wherein providing the composition comprises flowing the composition through a channel.
Example 3—the method according to Example 2, wherein the composition makes intimate contact with the sensor when flowing through the channel.
Example 4—the method according to Example 2, wherein the composition is separated from the sensor only by air when flowing through the channel.
Example 5—the method of Example 2 further comprising: positioning a light source substantially perpendicular relative to the channel.
Example 6—the method according to Example 5, wherein the light source is positioned perpendicular to the channel.
Example 7—the method according to Example 2 or Example 3, wherein the infrared light is emitted at an angle of incidence of about 20° to about 70°.
Example 8—the method according to Example 7, wherein the infrared light is emitted at an angle of about 90° relative to the flow of the composition through the channel.
Example 9—the method according to any foregoing Example, wherein the infrared light has a wavelength of from about 700 nm to about 1 mm, preferably 900 nm to about 5000 nm, preferably about 1000 nm to about 1500 nm.
Example 10—the method according to any foregoing Example, wherein the plurality of particles comprises an aluminum oxide, a magnesium oxide, a titanium oxide, a zinc oxide, a cerium oxide, or a combination of two or more thereof.
Example 11—the method according to any foregoing Example, wherein the plurality of particles comprises nanoparticles.
Example 12—the method according to any foregoing Example, wherein the plurality of particles have an average diameter of about 1 to about 800 nm, preferably about 100 to about 700 nm, preferably about 200 to about 600 nm, or preferably about 300 to about 550 nm.
Example 13—the method according to any foregoing Example further comprising: diluting the composition to obtain a concentration for the plurality of particles of about 0.1 to about 20 wt. %, preferably about 0.1 to about 15 wt. %, or preferably about 0.1 to about 10 wt. %, based on the total weight of the composition.
Example 14—the method according to any foregoing Example, wherein the composition further comprises an acid.
Example 15—the method according to Example 14, wherein the acid is at least one of: hydrochloric acid, hydrobromic acid, hydroiodic acid, nitric acid, perchloric acid, sulfuric acid, 1-glutamic acid, lactic acid, malic acid, succinic acid, acetic acid, formic acid, hydrogen sulfide, trichloracetic acid, fumaric acid, tartaric acid, citric acid, l-glutamic hydrochloride, and maleic acid.
Example 16—the method according to Example 14, wherein the acid is at least one of: 1-glutamic acid, lactic acid, hydrochloric acid, malic acid, succinic acid, acetic acid, formic, hydrogen sulfide, trichloracetic acid, fumaric acid, tartaric acid, citric acid, l-glutamic hydrochloride, and maleic acid.
Example 17—the method according to any one of Examples 14 to 16, wherein the acid is present in an amount from about 0.05 to about 15 wt. %.
Example 18—the method according to any foregoing Example, wherein the liquid carrier comprises water.
Example 19—a system comprising: a light source configured to emit an infrared light; a composition comprising a plurality of particles and a liquid carrier, wherein at least a portion of the plurality of particles is adapted to reflect infrared light; a sensor configured to sense the reflected infrared light; and an apparatus defining an inlet port, an exit port, and a channel extending therebetween, wherein the inlet port is configured to receive the composition and the outlet port is in fluid communication with the inlet port via the channel.
Example 20—the system according to Example 19, wherein the light source is positioned relative to the apparatus such that the infrared light is emitted at emitted at an angle of about 80° to about 100° relative to the composition when flowing through the channel.
Example 21—the system according to Example 19 or Example 20, wherein the light source is positioned relative to the apparatus such that the infrared light is emitted at an angle of about 90° relative to the composition when flowing through the channel.
Example 22—the system according to any one of Examples 19 to 21, wherein the sensor is positioned to be in intimate contact with the composition when the composition flows through the channel.
Example 23—the system according to any one of Examples 19 to 21, wherein the composition is separated from the sensor only by air when flowing through the channel.
Example 24—the system according to any one of Examples 19 to 23, wherein the plurality of particles comprises metal oxides selected from aluminum oxides, magnesium oxides, titanium oxides, zinc oxides, cerium oxides, and a combination of two or more thereof.
Example 25—the system according to any one of Examples 19 to 24, wherein the plurality of particles comprises titanium dioxide, alumina, or a combination thereof.
Example 26—the system according to any one of Examples 19 to 25, wherein the plurality of particles has an average diameter of about 1 to about 800 nm, preferably about 100 to about 700 nm, preferably about 200 to about 600 nm, or preferably about 300 to about 550 nm.
Example 27—the system according to any one of Examples 19 to 26, wherein the composition has a pH of about 1 to about 5, about 1 to about 4, about 1 to about 3, or about 1.5 to about 3.
Example 28—the system according to any one of Examples 19 to 27, wherein the composition further comprises an acid.
Example 29—the system according to Example 28, wherein the acid is at least one of: hydrochloric acid, hydrobromic acid, hydroiodic acid, nitric acid, perchloric acid, sulfuric acid, 1-glutamic acid, lactic acid, malic acid, succinic acid, acetic acid, formic acid, hydrogen sulfide, trichloracetic acid, fumaric acid, tartaric acid, citric acid, l-glutamic hydrochloride, and maleic acid.
Example 30—the system according to Example 28, wherein the acid is at least one of: 1-glutamic acid, lactic acid, hydrochloric acid, malic acid, succinic acid, acetic acid, formic, hydrogen sulfide, trichloracetic acid, fumaric acid, tartaric acid, citric acid, l-glutamic hydrochloride, and maleic acid.
Example 31—the system according to any one of Examples 28 to 30, wherein the acid is present in an amount from about 0.05 to about 15 wt. %.
Example 32—the system according to any one of Examples 19 to 31, wherein the liquid carrier comprises water.
Example 33—a calibration composition for calibrating an infrared reflectance device and/or sensor, the calibration composition comprising: a liquid carrier; a plurality of metal oxide particles; and an acid, wherein the calibration composition has a pH of about 1 to 7.
Example 34—the calibration composition according to Example 33, wherein the plurality of metal oxides has an average diameter of less than 1 μm.
Example 35—the calibration composition according to Example 34, wherein the plurality of metal oxides has an average diameter of about 1 to about 800 nm, preferably about 100 to about 700 nm, preferably about 200 to about 600 nm, or preferably about 300 to about 550 nm.
Example 36—the calibration composition according to any one of Examples 33 to 35, wherein at least one of the plurality of metal oxides is selected from aluminum oxides, magnesium oxides, titanium oxides, zinc oxides, cerium oxides, and a combination of two or more thereof.
Example 37—the calibration composition according to any one of Examples 33 to 36, wherein at least one of the plurality of metal oxides is selected from aluminum oxides, titanium oxides, and a combination of two or more thereof.
Example 38—the calibration composition according to any one of Examples 33 to 37, wherein the plurality of metal oxide particles comprises titanium dioxide, alumina, or a combination thereof.
Example 39—the calibration composition according to any one of Examples 33 to 37, wherein at least one of the plurality of metal oxides is selected from CeO₂, TiO₂, MgO, Al₂O₃, ZnO, and a combination of two or more thereof.
Example 40—the calibration composition according to any one of Examples 33 to 39, wherein the calibration composition has a pH of about 1 to about 5, about 1 to about 4, about 1 to about 3, or about 1.5 to about 3.
Example 41—the calibration composition according to any one of Examples 33 to 40 further comprising a stabilizing agent.
Example 42—the calibration composition according to Example 41, wherein the stabilizing agent comprises poly(methacrylic acid).
Example 43—the calibration composition according to Example 41 further comprising a stabilizing agent, wherein the calibration composition has a pH of about 2 to 7, about 3 to 7, about 4 to 7, about 4 to about 6.5, or about 5 to about 6.5.
Example 44—the calibration composition according to any one of Examples 33 to 43, wherein the liquid carrier comprises water.
Example 45—the calibration composition according to any one of Examples 33 to 43, wherein the calibration composition is an aqueous solution.
Example 46—the calibration composition according to any one of Examples 33 to 45, wherein the acid is at least one of: hydrochloric acid, hydrobromic acid, hydroiodic acid, nitric acid, perchloric acid, sulfuric acid, l-glutamic acid, lactic acid, malic acid, succinic acid, acetic acid, formic acid, hydrogen sulfide, trichloracetic acid, fumaric acid, tartaric acid, citric acid, 1-glutamic hydrochloride, and maleic acid.
Example 47—the calibration composition according to any one of Examples 33 to 45, wherein the acid is at least one of: l-glutamic acid, lactic acid, hydrochloric acid, malic acid, succinic acid, acetic acid, formic, hydrogen sulfide, trichloracetic acid, fumaric acid, tartaric acid, citric acid, l-glutamic hydrochloride, and maleic acid.
Example 48—the calibration composition according to any one of Examples 33 to 47, wherein the acid is present in an amount from about 0.05 to about 15 wt. %.

1. A method for calibrating an infrared reflectance device using a liquid composition, the method comprising:
providing a composition comprising a plurality of particles and a liquid carrier;
emitting an infrared light at the composition using a light source to produce a reflected light; and
sensing the reflected light from the composition using a sensor.

2. The method according to claim 1, wherein providing the composition comprises flowing the composition through a channel.

3. The method according to claim 2, wherein the composition makes intimate contact with the sensor when flowing through the channel.

4. The method according to claim 2, wherein the composition is separated from the sensor only by air when flowing through the channel.

5. The method of claim 2 further comprising:
positioning a light source substantially perpendicular relative to the channel.

6. The method according to claim 5, wherein the light source is positioned perpendicular to the channel.

7. The method according to claim 2, wherein the infrared light is emitted at an angle of incidence of about 20° to about 70°.

8. The method according to claim 7, wherein the infrared light is emitted at an angle of about 90° relative to the flow of the composition through the channel.

9. The method according to claim 1, wherein the infrared light has a wavelength of from about 700 nm to about 1 mm, preferably 900 nm to about 5000 nm, preferably about 1000 nm to about 1500 nm.

10. The method according to claim 1, wherein the plurality of particles comprises an aluminum oxide, a magnesium oxide, a titanium oxide, a zinc oxide, a cerium oxide, or a combination of two or more thereof.

11. The method according to claim 1, wherein the plurality of particles comprises nanoparticles.

12. The method according to claim 1, wherein the plurality of particles have an average diameter of about 1 to about 800 nm, preferably about 100 to about 700 nm, preferably about 200 to about 600 nm, or preferably about 300 to about 550 nm.

13. The method according to claim 1, further comprising:
diluting the composition to obtain a concentration for the plurality of particles of about 0.1 to about 20 wt. %, preferably about 0.1 to about 15 wt. %, or preferably about 0.1 to about 10 wt. %, based on the total weight of the composition.

14. The method according to claim 1, wherein the composition further comprises an acid.

15. The method according to claim 14, wherein the acid is at least one of: hydrochloric acid, hydrobromic acid, hydroiodic acid, nitric acid, perchloric acid, sulfuric acid, I-glutamic acid, lactic acid, malic acid, succinic acid, acetic acid, formic acid, hydrogen sulfide, trichloracetic acid, fumaric acid, tartaric acid, citric acid, I-glutamic hydrochloride, and maleic acid.

16. The method according to claim 14, wherein the acid is at least one of: I-glutamic acid, lactic acid, hydrochloric acid, malic acid, succinic acid, acetic acid, formic, hydrogen sulfide, trichloracetic acid, fumaric acid, tartaric acid, citric acid, l-glutamic hydrochloride, and maleic acid.

17. The method according to claim 14, wherein the acid is present in an amount from about 0.05 to about 15 wt. %.

18. The method according to claim 1, wherein the liquid carrier comprises water.

19. A system comprising:
a light source configured to emit an infrared light;
a composition comprising a plurality of particles and a liquid carrier, wherein at least a portion of the plurality of particles is adapted to reflect infrared light;
a sensor configured to sense the reflected infrared light; and
an apparatus defining an inlet port, an exit port, and a channel extending therebetween, wherein the inlet port is configured to receive the composition and the outlet port is in fluid communication with the inlet port via the channel.

20. The system according to claim 19, wherein the light source is positioned relative to the apparatus such that the infrared light is emitted at emitted at an angle of about 80° to about 100° relative to the composition when flowing through the channel.

21. The system according to claim 19, wherein the light source is positioned relative to the apparatus such that the infrared light is emitted at an angle of about 90° relative to the composition when flowing through the channel.

22. The system according to claim 19, wherein the sensor is positioned to be in intimate contact with the composition when the composition flows through the channel.

23. The system according to claim 19, wherein the composition is separated from the sensor only by air when flowing through the channel.

24. The system according to claim 19, wherein the plurality of particles comprises metal oxides selected from aluminum oxides, magnesium oxides, titanium oxides, zinc oxides, cerium oxides, and a combination of two or more thereof.

25. The system according to claim 19, wherein the plurality of particles comprises titanium dioxide, alumina, or a combination thereof.

26. The system according to claim 19, wherein the plurality of particles has an average diameter of about 1 to about 800 nm, preferably about 100 to about 700 nm, preferably about 200 to about 600 nm, or preferably about 300 to about 550 nm.

27. The system according to claim 19, wherein the composition has a pH of about 1 to about 5, about 1 to about 4, about 1 to about 3, or about 1.5 to about 3.

28. The system according to claim 19, wherein the composition further comprises an acid.

29. The system according to claim 28, wherein the acid is at least one of: hydrochloric acid, hydrobromic acid, hydroiodic acid, nitric acid, perchloric acid, sulfuric acid, I-glutamic acid, lactic acid, malic acid, succinic acid, acetic acid, formic acid, hydrogen sulfide, trichloracetic acid, fumaric acid, tartaric acid, citric acid, l-glutamic hydrochloride, and maleic acid.

30. The system according to claim 28, wherein the acid is at least one of: I-glutamic acid, lactic acid, hydrochloric acid, malic acid, succinic acid, acetic acid, formic, hydrogen sulfide, trichloracetic acid, fumaric acid, tartaric acid, citric acid, l-glutamic hydrochloride, and maleic acid.

31. The system according to claim 28, wherein the acid is present in an amount from about 0.05 to about 15 wt. %.

32. The system according to claim 19, wherein the liquid carrier comprises water.
